(19) United States
(12) Patent Application Publication
(10) Pub. No.: US 2005/0112558 A1
(43) Pub. Date: May 26, 2005

(54) Prognostic PCR assay for severe acute respiratory syndrome (SARS)

(75) Inventors: Yuk Ming Dennis Lo, Kowloon (HK); Kai On Ng, Shatin (HK); Kwan Chee Allen Chan, Kowloon (HK); Wai Kwun Rossa Chiu, Tai Po (HK); Siu Lun John Tam, Shatin (HK); Chi Wan Emily Hung, Tsing Yi (HK); Siu Chung Stephen Chim, Wan Chai (HK); Yu Kwan Tong, Tuen Mun (HK)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Chinese University of Hong Kong, Kowloon (HK)

(21) Appl. No.: 10/948,915

(22) Filed: Sep. 23, 2004

Related U.S. Application Data
(60) Provisional application No. 60/505,896, filed on Sep. 24, 2003.

(51) Int. Cl. C12Q 1/70; C12Q 1/68; C07H 21/04

(52) U.S. Cl. 435/5; 435/6; 536/23.72

(57) ABSTRACT

Disclosed are nucleic acid primers, probes and standard target sequences that allow for the quantitative assessment of the SARS-CoV viral titer found in a biological specimen from a patient. Quantifying the viral titer in the patient sample allows the practitioner to make a prognostic determination of the severity of the SARS-CoV infection and the necessary treatment regime.

### CROSS-REFERENCES TO RELATED APPLICATIONS

THIS APPLICATION IS A CONTINUATION-IN-PART APPLICATION CLAIMING THE BENEFIT OF PROVISIONAL APPLICATION Ser. No. 60/505,896, FILED Sep. 24, 2003, THE DISCLOSURE OF WHICH IS INCORPORATED HEREIN BY REFERENCE.

### BACKGROUND OF THE INVENTION

1. Field of the Invention

Disclosed are nucleic acid primers, probes and standard target sequences that allow for the quantitative assessment of the SARS-CoV viral titer found in a biological specimen from a patient. Quantifying the viral titer in the patient sample allows the practitioner to make a prognostic determination of the severity of the SARS-CoV infection and the necessary treatment regime.

2. Description of Related Art

Severe acute respiratory syndrome (SARS) has recently emerged as an infectious disease caused by a novel coronavirus, the SARS-coronavirus (SARS-CoV) (Drosten C, et al., N Engl J Med 2003;348:1967-76; Ksiazek T G, et al. N Engl J Med 2003;348:1953-66; Fouchier R A, et al., Nature 2003;423:240; and Peiris J S, et al., Lancet 2003;361:1319-25). Thus far, molecular testing for SARS has mainly been focused on reverse transcriptase polymerase chain reaction (RT-PCR) analysis of nasopharyngeal aspirates, urine and stools (Poon L L, et al., Clin Chem 2003;49:953-5; and Peiris J S, et al., Lancet 2003;361:1767-72). However, the quantitative interpretation of these data is difficult due to the inability to standardize the expression of such data as a result of the influence of numerous factors, such as sampling technique for nasopharyngeal aspirates, urine volume, variations of bowel transit time (e.g. during diarrhea) or the stool consistency.

To date there is a paucity of data concerning the detection of SARS-CoV in the plasma/serum of SARS patients. There has been a single report showing the relatively low sensitivity of detecting SARS-CoV RNA in plasma using an ultracentrifugation-based approach, with low concentrations of SARS-CoV detected in the plasma of a patient 9 days after disease onset (Drosten C, et al., N Engl J Med 2003;348:1967-76).

### BRIEF SUMMARY OF THE INVENTION

Based on publicly released full genomic sequences of SARS-CoV (Tsui S K W, et al., N Engl J Med 2003;349:187-188; Marra M A, et al., Science 2003;300:1399-404; Rota P A, et al., Science 2003;300:1394-9), we have developed real-time reverse transcriptase polymerase chain reaction (RT-PCR) assays specifically targeting two different regions of the SARS-CoV genome. In this study, we investigated if SARS-CoV RNA can be detected in serum and plasma samples during the early stage of SARS and to study the potential prognostic implications of such an approach. Preferably these samples are not centrifuged.

The present invention provides primers for detection and prognostic evaluation of SARS-CoV infection. Particularly, the primers are specific for the polymerase and nucleocapsid genes of the SARS-CoV. The invention also provides probes specific for each primer set and standard target nucleic acids that are recognized by the probes of the invention. Using the specific probes and standard target nucleic acids according to the methods of the present invention allows for quantitative determination of viral titer in a sample. The viral titer provides information allowing the practitioner to make a prognostic evaluation of the infected individual providing the sample.

Accordingly, the present invention provides a primer pair comprising a first primer having at least 10 contiguous nucleotides more preferably 16 nucleotides in length with a nucleotide sequence at least 75%, preferably at least 85%, more preferably 90%, most preferably 95%, 96%, 97%, 98%, or 99%, ideally 100% identical to an oligonucleotide A, or fragment thereof, and hybridizing under stringent conditions to a reference oligonucleotide complementary to oligonucleotide A; and, a second primer having at least 10 contiguous nucleotides more preferably 16 nucleotides in length with a nucleotide sequence at least 75%, preferably at least 85%, more preferably 90%, most preferably 95%, 96%, 97%, 98%, or 99%, ideally 100% identical to an oligonucleotide B, or fragment thereof, and hybridizing under stringent conditions to a reference oligonucleotide complementary to oligonucleotide B. Oligonucleotide A and oligonucleotide B are selected from the group consisting of SEQ ID NO:1 and 2; SEQ ID NO:4 and 5; SEQ ID NO:7 and SEQ ID NO:8; and SEQ ID NO:10 and 11, making up the primer set. In one aspect at least one primer of the primer set has at least 16 nucleotides.

Another embodiment of the present invention is a method for detecting the presence of SARS-CoV in a sample. The method involves contacting the sample with a primer pair as described above. By performing RT-PCR (reverse transcriptase PCR) on the sample, to amplify a SARS-specific nucleic acid, preferably a nucleic acid encoding the SARS polymerase or nucleocapsid, or a fragment thereof. The presence or absence of the amplified SARS-CoV nucleic acid is then determined, with the presence of the nucleic acid being indicative of the presence of SARS-CoV in the sample. The method is particularly suitable for screening populations of individuals to determine patterns of infection and treatment strategies. Preferred aspects of the method include SARS-specific probes to the SARS-specific nucleic acid. Preferably, the SARS-specific probe comprises at least 10 contiguous nucleotides selected from the group of nucleotide sequences consisting of SEQ ID NO:3, SEQ ID NO:6, SEQ ID NO:9 and SEQ ID NO:12.

In preferred aspects of the method, the SARS-specific probes are matched to the primer sets. For example, when the first primer nucleotide sequence is SEQ ID NO:1 or a fragment of at least 10 contiguous nucleotides thereof and the second primer nucleotide sequence is SEQ ID NO:2 or a fragment of at least 10 contiguous nucleotides more preferably 16 contiguous nucleotides thereof; determination of the presence of a SARS-specific nucleic acid may be performed by hybridizing at least 10 contiguous nucleotides more preferably 16 contiguous nucleotides of SEQ ID NO:3 to the SARS-specific nucleic acid. Similarly, when the first primer nucleotide sequence is SEQ ID NO:4 or a fragment of at least 10 contiguous nucleotides more preferably 16 contiguous nucleotides thereof and the second primer nucleotide sequence is SEQ ID NO:5 or a fragment of at least 10 contiguous nucleotides more preferably 16 contiguous nucleotides thereof, the probe used in determining the presence of a SARS-specific nucleic acid comprises at least 10 contiguous nucleotides more preferably 16 contiguous nucleotides of SEQ ID NO:6. For the primer set of SEQ ID NO:7 or a fragment of at least 10 contiguous nucleotides more preferably 16 contiguous nucleotides thereof and a second primer nucleotide sequence of SEQ ID NO:8 or a fragment of at least 10 contiguous nucleotides more preferably 16 contiguous nucleotides thereof; the corresponding probe is at least 10 contiguous nucleotides more preferably 16 contiguous nucleotides of SEQ ID NO:9. Finally, for the primer set of SEQ ID NO:10 or a fragment of at least 10 contiguous nucleotides more preferably 16 contiguous nucleotides thereof and a second primer of SEQ ID NO:11 or a fragment of at least 10 contiguous nucleotides more preferably 16 contiguous nucleotides thereof; the corresponding probe is at least 10 contiguous nucleotides more preferably 16 contiguous nucleotides of SEQ ID NO:12.

An additional embodiment of the invention is a method of performing prognostic testing on an individual infected with SARS-CoV. This method includes: (a) contacting a plasma sample from the individual with a SARS-specific primer pair, as described above; (b) performing RT-PCR on the sample wherein, if present, a SARS-specific target nucleic acid is amplified; (c) determining a plasma SARS-CoV concentration by quantifying the amplification of the SARS-specific target nucleic acid and comparing the quantity with a standard calibration curve; and, (d) providing a prognosis based on the plasma SARS-CoV concentration. With automated equipment, as described herein, this embodiment of the invention is suitable for screening populations of individuals suspected of being infected with SARS-CoV, and arriving at a prognostic indication for each infected individual identified.

Some aspects of the prognostic method use plasma samples formed by clotting blood samples of individuals suspected of being infected with the SARS virus.

Other aspects of the method use standard target nucleic acids to construct the standard calibration curve. Preferably, these standard target nucleic acids have custom nucleotide sequences designed for use with specific primer pairs. Typically the standard calibration curve is constructed using one or more isolated single-stranded nucleic acids selected from the group consisting of SEQ ID NO:13, 14, 15 and 16. A serial dilution of these nucleic acids is performed to yield a standard calibration curve with a range from about 1 to 10¹⁰, more preferably from about 10 to 10⁷ replicons/mL. Similarly, detection of the SARS-specific nucleic acid in the sample may be performed using probes that are matched to specific primer pairs, as described above.

Another embodiment of the invention is kits for the detection of the presence of SARS-CoV in a sample. Kits of the invention include a primer set, as described above, and methods for their use. Kits may also contain probes or standard target nucleic acids that are preferably customized for use with the particular primer set of the kit. Preferred primer set probe combinations are discussed above. Preferred standard target nucleic acids are discussed in detail below.

Other embodiments of the invention include isolated nucleic acids of at least 10 nucleotides more preferably 16 nucleotides and comprising a nucleotide sequence at least 85%, more preferably 90%, most preferably 95%, 96%, 97%, 98%, or 99%, ideally 100% complementary to 10 or more contiguous nucleotides of a nucleotide sequence selected from the group consisting of SEQ ID NO: 1, 2, 3, 4, 5, 6, 7, 8, 9, 10, 11 and 12 or fragment thereof, wherein the isolated nucleic acid hybridizes to a SARS-CoV target, but not other coronavirus types, under selectively stringent conditions. These isolated nucleic acids find utility in blotting techniques for identifying SARS-CoV nucleic acids. Preferably nucleic acids for such techniques have a nucleotide sequence is selected from the group consisting of SEQ ID NO: 1, 2, 3, 4, 5, 6, 7, 8, 9, 10, 11 and 12.

### BRIEF DESCRIPTION OF THE DRAWINGS

FIG. 1 depicts the levels of SARS-CoV in the serum of patients who required admission to the intensive care unit (ICU) and not required admission to the intensive care unit (non-ICU). The data were generated using the RT-PCR system described in Example 1.

FIG. 2 diagrams the serum SARS-CoV RNA concentrations in SARS patients on the day of hospital admission. Box plot of SARS-CoV concentrations (common logarithmic scale) in sera of SARS patients requiring and not requiring ICU admission. The unfilled boxes denote the SARSPol1 system while the shaded boxes denote the SARSN system. The lines inside the boxes denote the medians. The boxes mark the interval between the 25^{th} and 75^{th} percentiles. The whiskers denote the interval between the 10^{th} and 90^{th} percentiles. The filled circles mark the data points outside the 10^{th} and 90^{th} percentiles.

FIG. 3 illustrates serial analysis of plasma SARS-CoV RNA concentrations in pediatric SARS patients. Plots of plasma SARS-CoV RNA concentrations (copies of SARS-CoV RNA per mL of plasma) (Y-axis) against time after the onset of fever (day 1 refers the day of fever onset) (X-axis). The duration of fever and the periods of steroid and ribavirin treatment are indicated for each case. The arrows in patient 1 and patient 7 indicate the time of intravenous methylprednisolone treatment.

FIG. 4 correlates serum SARS-CoV RNA concentration between SARSPol1 and SARSN RT-PCR systems.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“SARS-CoV” refers to a member of the coronavirus family that is the causative agent of Severe Acute Respiratory Syndrome.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-o-methyl ribonucleotides and peptide-nucleic acids (PNAs).

Unless otherwise indicated, a particular nucleic acid sequence also implicitly encompasses conservatively modified variants thereof (e.g., degenerate codon substitutions, see below) and complementary sequences, as well as the sequence explicitly indicated. The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, 65%, 70%, 75%, 80%, preferably 85%, 90%, 91%, 92%, 93%, 94%, 95%, 96%, 97%, 98%, 99% or higher identity to an amino acid sequence such as SEQ ID NO:2 or a nucleotide sequence such as SEQ ID NO:1 or SEQ ID NO:3), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “stringent hybridization conditions” (or “stringent conditions”) refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

“SARS-specific nucleic acid” is a nucleic acid sequence present only in strains of the SARS-CoV coronavirus.

“SARS-specific probe” refers to an isolated nucleic acid that specifically hybridizes to a SARS-specific nucleic acid under stringent hybridization conditions.

“Duplex molecules” refers to antiparallel nucleic acids that hybridize over at least 75%, more preferably 80%, 90% 92%, 95%, most preferably 97%, 98%, or 99% and ideally 100% of the length of the shortest hybridizing nucleic acid, “hybridization” or “hybrisizing” being defined as normal Watson-Crick base-pairing between opposing nucleotides of each strand.

“Specifically hybridizing” refers to the interaction between two nucleic acids orientated antiparallel to each other and undergoing Watson-Crick base pairing over at least 75%, more preferably 80%, 90% 92%, 95%, most preferably 97%, 98%, or 99% and ideally 100% of the length of the shortest hybridizing nucleic acid under stringent hybridization conditions.

“Other coronavirus-specific nucleic acids” refers to nucleic acids that are specific to coronaviruses other than SARS-CoV.

The terms “complementary” or “complementarity” refer to polynucleotides (i.e., a sequence of nucleotides) related by base-pairing rules. For example, the sequence “5′-AGT-3′,” is complementary to the sequence “5′-ACT-3′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance for methods that depend upon binding between nucleic acids.

### INCORPORATION BY REFERENCE

All publications and patent applications cited in the specification are herein incorporated by reference as if each individual publication or patent application were specifically and individually indicated to be incorporated by reference.

### DETAILED DESCRIPTION OF THE INVENTION

I. Introduction

Based on our recent release of the complete genomic sequences of the SARS-coronavirus (GenBank accession number AY278554 and AY282752; http://www.ncbi.nlm.nih.gov/), we have developed real-time Reverse transcriptase polymerase chain reaction (RT-PCR) assays specifically targeting different regions of the SARS-CoV genome. The assays were able to detect as low as 5 copies of calibration targets using specific primers suitable for use in polymerase chain reaction (PCR) and reverse transcriptase polymerase chain reaction (RT-PCR). Probes for detecting the PCR products are also provided, as are standard target nucleic acids that are specifically designed for use with the primers of the invention, and provide a means for quantitating PCR results, as described herein.

Structurally, the nucleic acids of the present invention are defined by the nucleotide sequences presented herein. Functionally, the nucleic acids of the present invention specifically recognize SARS-CoV, but not other coronaviruses, or are recognized by primers that specifically recognize SARS-CoV but not other coronaviruses.

II. Construction of Nucleic Acids Used in the Invention

Nucleic acid primers and probes of the present invention may be prepared using techniques well known to those of skill in the art. For example, nucleic acids may be synthesized chemically, e.g., according to the solid phase phosphoramidite triester method first described by Beaucage & Caruthers, Tetrahedron Letts., 22:1859-1862 (1981), using an automated synthesizer, as described in Van Devanter et. al., Nucleic Acids Res., 12:6159-6168 (1984). Oligonucleotides may purified, e.g., by native acrylamide gel electrophoresis or by anion-exchange HPLC as described in Pearson & Reanier, J. Chrom., 255:137-149 (1983).

For example, using the nucleotide sequences supplied herein, one of skill in the art can synthesize a nucleic acid primer or probe that is at least 10, more preferably at least 16 contiguous nucleotides in length having at least 75%, more preferably 80%, 85% or 90%, most preferably 95%, 96%, 97%, 98%, or 99%, ideally 100% identity to a contiguous nucleotide sequence present in SEQ ID NO:1-16.

Nucleic acids that are at least 10 nucleotides more preferably 16 nucleotides in length with a nucleotide sequence at least 85%, more preferably 90%, most preferably 95%, 96%, 97%, 98%, or 99%, ideally 100% complementary to 10 or more contiguous nucleotides of SEQ ID NO: 1, 2, 3, 4, 5, 6, 7, 8, 9, 10, 11 or 12 may also be synthesized using the disclosed techniques. Such nucleic acids are functionally characterized by their ability to hybridize to a SARS-specific nucleic acid, but not nucleic acids specific to other coronaviruses under selectively stringent conditions.

Synthesized nucleic acids having the desired sequence may be amplified using well known techniques. See, e.g., Sambrook, J., Fritsch, E. F., and Maniatus, T., Molecular Cloning, A Laboratory Manual 2nd ed. (1989); Kriegler, Gene Transfer and Expression: A Laboratory Manual (1990); and Current Protocols in Molecular Biology (Ausubel et al., eds., 1994). The nucleotide sequence of isolated nucleotides of the invention may be confirmed using automated sequencing techniques well known in the art.

In some embodiments, nucleic acids of the invention may include modified bases as described in Uhlmann, et al. (1990, Chemical Reviews 90: 543-584). Preferred nucleotide analogs are unmodified G, A, T, C and U nucleotides; pyrimidine analogs with lower alkyl, alkynyl or alkenyl groups in the 5 position of the base and purine analogs with similar groups in the 7 or 8 position of the base. Other preferred nucleotide analogs are 5-methylcytosine, 5-methyluracil, diaminopurine, and nucleotides with a 2′-O-methylribose moiety in place of ribose or deoxyribose. As used herein lower alkyl, lower alkynyl and lower alkenyl contain from 1 to 6 carbon atoms and can be straight chain or branched. These groups include methyl, ethyl, propyl, isopropyl, butyl, isobutyl, tertiary butyl, amyl, hexyl and the like. A preferred alkyl group is methyl.

The sequence of isolated nucleic acids may be verified after using, e.g., the chain termination method for sequencing double-stranded templates of Wallace et al., Gene, 16:21-26 (1981) or using the chemical degradation method of Maxam and Gilbert (1980) in Grossman and Moldave (eds.) Academic Press, New York, Methods in Enzymology 65:499-560. Sequences of short oligonucleotides can also be analyzed by laser desorption mass spectroscopy or by fast atom bombardment (McNeal, et al., 1982, J. Am. Chem. Soc. 104: 976; Viari, et al., 1987, Biomed. Enciron. Mass Spectrom. 14: 83; Grotjahn et al., 1982, Nuc. Acid Res. 10: 4671). Analogous sequencing methods are available for RNA.

III. Suitable Biological Samples

Samples suitable for analysis using the methods of the present invention may be taken from any source of SARS-CoV nucleic acid, including feces, saliva and nasal discharge. Preferred samples of the invention are taken from blood, or blood fractions. Using the methods and nucleic acids described herein, one of skill in the art may detect both DNA and RNA forms of SARS-CoV, including viral, proviral, and mRNA in such samples.

Samples may be prepared by separating nucleic acids from other cellular material by, for example, centrifugation or precipitation. Blood, for example, may be clotted followed by removal of cellular material (and the clot) by centrifugation at 8,000×g for 10 minutes. A preferred method of isolating cell-bound nucleic acids includes lysing the cells with a salt or detergent solution. Cellular material may be removed by centrifugation, or the nucleic acid precipitated using techniques well known to those of skill in the art. However, an advantageous aspect of the present invention is the ability to assay a patient's blood sample without subjecting the sample to centrifugation. By removing the centrifugation step, throughput of samples is increased, allowing for processing of a greater number of samples in a given time. This in turn allows for earlier diagnosis and treatment of the disease.

More elaborate nucleic acid preparations may also be employed, for example successive phenol/chloroform extractions of a preparation to remove protein and lipid, followed by ethanol precipitation of the nucleic acid. Such techniques are well-known to those of skill in the art.

Prepared samples are preferably diluted in a suitable buffer to standardize the initial nucleic acid concentration. The desired nucleic acid concentration is dependent upon the sample and the method used, and is easily obtained through routine experimentation following procedures known in the art (See, e.g., PCR Protocols: A Guide to Methods and Applications (Innis et al., eds, 1990)).

IV. PCR-Based Assays

The present invention provides nucleic acid primers and probes for the detection of SARS-CoV from a biological sample. When used in the methods of the present invention, these nucleic acids allow for the quantitative determination of viral load in the sample, which can be used to determine the prognosis of the individual supplying the sample. Although the nucleic acids of the present invention are useful in applications that directly detect viral nucleic acids (e.g., Northern and Southern blotting assays), their preferred use is in PCR protocols, most preferably quantitative PCR protocols. Both quantitative and non-quantitative PCR protocols are well known in the art and discussed at length in available literature. See, e.g., PCR Protocols: A Guide to Methods and Applications (Innis et al., eds, 1990)). Such methods can be used to PCR amplify the SARS-CoV virus, provirus, mRNAs hybridizing to the nucleic acids of the invention and DNA libraries containing SARS-CoV nucleic acid. Exemplary methods demonstrating working procedures of the present invention are described in detail in the examples section, below.

A. Amplifying Target Nucleic Acids

The present invention describes methods for determining the prognosis of a patient based on the SARS-CoV viral titer found in a patient sample, preferably a patient's blood sample. Viral titer of a sample may be determined using the nucleic acid primers and probes of the present invention with any one of a number of quantitative or semi-quantitative PCR methods known in the art or described herein. For example, Gilliland describes a quantitative PCR method based on a competitive PCR (PNAS (1990) 87:2725). To determine DNA amounts, he uses a dilution series of an internal standard that is amplified simultaneously with the sample. The PCR is carried out until saturation. This also allows for the detection of the PCR products by means of ethidium bromide staining. Subsequently, the PCR products are separated on a gel, the number of copies of the sample is compared to the number of copies of the standard dilution series, and thus the concentration of the sample is estimated. This concentration estimate will be exact if the concentration of the standard and the sample have been amplified in a reaction vessel approximately at a ratio of 1:1. This, in turn, implies that the determination of the DNA amount will be the more precise, the more standard dilutions are used. In principle, nucleic acid amplification means methods based on the technology developed by Mullis et al. (U.S. Pat. Nos. 4,683,195 and 4,683,202), and others, e.g. the polymerase chain reaction (PCR), the reverse transcriptase-PCR (RT-PCR) of the ligase-CR (LCR).

Another method of quantitating RNA has been suggested by Wang et al. (PNAS (1989) 86:9717). This PCR method is stopped in the exponential phase. By amplifying various standard concentrations, the authors provide a calibration curve (external standardization). Since the number of PCR products increases at a rate directly proportional to the number of RNA molecules present in the exponential reaction phase, this calibration curve is a straight line in which one can read off the concentration of an amplified sample. A disadvantage of this method is that the final concentration of the PCR products is relatively low so that sensitive detection methods must be used for the detection. Wang et al. use radioactively labelled nucleotides.

A PCR-based method adaptable for use with nucleic acids of the present invention is described by Porcher et al. (BioTechnique 13 (1992), 106). Porcher et al. succeeded in improving the detection of small amounts of PCR products by employing fluorescence-labeled primers and quantitating the PCR products with an automatic laser fluorescence DNA sequencer.

Single-primer extension may be directed by a thermophilic DNA polymerase, usually a 3′-5′ exonuclease-minus derivative polymerase, e.g. Vent™ (exo-) DNA polymerase. Multiple copies of the target(s) are generated during the extension reaction, in which repeated cycles of denaturation, oligonucleotide primer annealing, and DNA polymerase-directed primer extension are performed. Following generation of multiple single-stranded copies of target(s) from the cDNA pool, the complementary strands are generated. In one embodiment, generation of the complementary strand is mediated by a common primer that binds to all amplified targets.

All these methods enable the determination of small amounts of nucleic acids homologous with nucleic acids of the present invention, such as the polymerase and nucleocapsid genes, SARS-CoV genomic and proviral nucleic acids, and polymerase and nucleocapsid mRNAs.

In general, the methods of the present invention include linearly amplifying a target nucleic acid (or multiple nucleic acids) of SARS-CoV using a quantitative PCR method and the primers of the present invention. As provided, these primers are specific for polymerase (pol) (SEQ ID NO:1 and 2, or 4 and 5) and nucleocapsid (nuc) (SEQ ID NO: 7, 8) nucleotide sequences. In addition to the primers described in the examples of the present application, any subsequences of at least 10, more preferably, 11, 12 or 13, most preferably 14 or 15, and ideally at least 16 contiguous nucleotides selected from the provided primer sequences (SEQ ID NO:1, 2, 4, 5, 7 or 8) may be used as a substitute for the full length primer. In a preferred embodiment of the method according to the invention, the amplification step is stopped while in the exponential phase.

As described below, standard target nucleic acids (e.g., SEQ ID NO.:13-16) are amplified in parallel. The amplified standard nucleic acids are treated identically to the amplified sample target nucleic acids. For quantitative analysis, the standard target nucleic acids are serially diluted prior to amplification. Serial dilution allows the practitioner to prepare a calibration curve, allowing quantification of the target nucleic acid in the sample. As noted, the present invention is suitable for detecting both DNA and RNA target nucleic acids using standard or RT-PCR techniques well-known to those of skill in the art.

B. Detection of the Amplified Target Nucleic Acid.

The amplified target nucleic acid may be detected by any suitable manner known in the art. The determination of the nucleic acid amounts (the quantity of DNA is provided in the form of a mass or as the number of copies of a certain nucleic acid molecule) after the amplification may be determined by any suitable manner. Detection may optionally include a step for enriching the amplified target nucleic acid prior to detection. Preferably, this enrichment step includes gel electrophoresis or a chromatographic method. The probes used in the detection procedure preferably contain groups which increase the detection limit of the amplified nucleic acids, e.g., fluorescent or radioactive groups or chemical groups which can be detected by means of affinity proteins and subsequent detection reactions (e.g., Biotin-Avidin, Digoxigenin labeling etc.), primers containing fluorescent groups being particularly preferred.

Exemplary detection methods include binding amplified target nucleic acid to an appropriately immobilized complementary sequence followed by detection by plasmon resonance. Semi-quantitative results may be obtained through solid-phase sequencing. A preferred embodiment uses labeled probes, for example radiolabelled or fluorescently-labeled nucleotide probes that specifically hybridize with SARS-CoV nucleic acid sequences. Particularly preferred embodiments use nucleic acid probes that are doubly labeled with fluorescent molecules. Preferred fluorescent labels include tetramethylrhodamine (TAMRA), rhodamine (ROX), FluorePrime manufactured by Pharmacia and ‘FAM’, which is a fluorescein dye incorporated into the nucleic acid during synthesis using the reagent 6-FAM phosphoramidite (Perkin-Elmer Biosystems). Other methods of detecting and quantifying amplified target nucleic acids may also be used, as will be evident to those of skill in the art.

Detection methods may be automated combining, for example, probe application and detection. Preferred embodiments include a nucleic acid detection device, preferably a fluorescence-sensitive nucleic acid detection device. Examples of such nucleic acid detection devices include flourimeters, automatic DNA sequencers with laser-induced fluorescence measuring devices (e.g. Gene Scanner™ 373A of Applied Biosystems) and HPLC-devices. A particular advantage of the Gene Scanner is that it is possible to differentiate between different fluorescence dyes in one single lane. This allows for the simultaneous processing of a plurality of samples on one gel, since all lanes available on the gel may be used for samples. Furthermore, it is possible to analyze a plurality of PCR products, labeled with different fluorescence dyes, in one single lane (multiplex-PCR), and thereby to detect genomic DNA of various origins in a sample. When simultaneously detecting two different nucleic acids, e.g., in one sample, furthermore expenditures and costs are nearly cut in half. Automated detection and quantitation of amplified products is particularly useful when screening large numbers of samples, for example when screening a population of individuals during an outbreak of SARS.

C. Quantifying Target Nucleic Acids and Prognostic Implications.

The present invention also provides methods for performing prognostic testing on individuals infected with SARS-CoV. Prognostic testing according to the invention is based on the PCR methods for detecting SARS-CoV target nucleic acids described above. Accuracy of the prognostic diagnosis is dependent upon the accuracy of the viral titer determination, as determined by the quantitative procedures described herein.

Quantitative analysis by PCR is preferably performed using standard “target” nucleic acids, preferably amplified in parallel with the sample target nucleic acids, more preferably amplified in the same reaction mixture as the sample target nucleic acids. Preferably the standard target nucleic acid concentration is close to or matches the sample target nucleic acid concentration prior to amplification. One method of utilizing the standard target nucleic acids of the invention is by making serial dilutions. By assaying the standards at a number of different starting concentrations, the likelihood of producing a standard with a nucleic acid concentration at or near that of the sample target nucleic acid is increased. Generally, the closer the sample target nucleic acid concentration is to a standard target nucleic acid concentration, the greater the accuracy of the assay. A preferred method of using the standard target nucleic acids is by plotting a regression curve using a serial dilution series, as discussed above. Once constructed, the concentration of the sample target nucleic acid may be read from the curve. For example, when fluorescent probes are used to detect the amplified target nucleic acids, serial dilution series of standard target nucleic acids may be used to construct a standard plot of fluorescent intensity versus nucleic acid concentration. The concentration of an unknown sample target nucleic acid can then be determined by comparing the fluorescent intensity of the probe bound to the sample target with the standard plot. For convenience, the nucleic acid concentration in such standard plots is expressed in molar values or replicons/μl. Standard plots of this type can cover a wide range of nucleic acid concentrations, typically between 10¹ and 10⁷ replicons/μl.

To ensure accurate quantification of target nucleic acids, it is preferable to use the same primers for standard and sample target nucleic acids. Primers should possibly be 100% homologous to the primer binding sites. Therefore, particularly preferred probes of the present invention are necessarily paired with specific primers. For example, primer pairs taken from the nucleotide sequences of SEQ ID NO:1 and 2 are used with probes taken from the nucleotide sequence of SEQ ID NO:3. Similarly primer pairs taken from the nucleotide sequences of SEQ ID NO:4 and 5 are used with probes taken from the nucleotide sequence of SEQ ID NO:6; primer pairs taken from the nucleotide sequences of SEQ ID NO:7 and 8 are used with probes taken from the nucleotide sequence of SEQ ID NO:9; and primer pairs taken from the nucleotide sequences of SEQ ID NO:10 and 11 are used with probes taken from the nucleotide sequence of SEQ ID NO:12.

Using the quantitative assays of the present invention, the prognosis of a patient infected with SARS-CoV may be ascertained from the viral titer determined. For example, a viral titer of under 1000 copies/mL, preferably under 750 copies/mL, most preferably under 500 copies/mL of plasma or serum or whole blood is indicative of a good prognosis with an excellent chance of full recovery from the SARS infection. In contrast, a viral titer of greater than 15000, more preferably greater than 16000, better still greater than 17000 and ideally greater than 20000 copies/mL of plasma or serum or whole blood indicates that the patient is likely to have severe symptoms that are likely to require intensive care and may result in death.

For a review of quantitative PCR procedures see Reischl, U. and Kochanowski, B. (1999) “Quantitative PCR” Quantitative PCR Protocols (pp.3-30). Humana Press., Totowa, N.J.; and also Ferre F. (1992) Quantitative or semi-quantitative PCR: Reality versus myth. PCR Methods Appl. 2, 1-9.)

V. Alternative Assays

In addition to the sensitive PCR techniques described above, the probes and primers of the present invention also find utility in other quanitative and semi-quantitative assay techniques designed to detect small amounts of nucleic acids in a sample. Several alternative techniques are known by those of skill in the art and include NASBA, LAMP and Branched DNA signal amplification. Each of these exemplary techniques are described in greater detail, below.

NASBA is an isothermal method of nucleic acid amplification for detecting specific nucleic acid sequences. The method is highly suited for the amplification of RNA. Nucleic acids are isolated from a cellular source by lysing the cell with guanidine thiocyanate and Triton X-100. The lysis buffer is removed and the nucleic acid purified by chromatographic methods using silicon dioxide particles.

The nucleic acid is amplified in NASBA through the coordinated activities of three enzymes, AMV Reverse Transcriptase, RNase H, and T7 RNA Polymerase. Quantitative detection is achieved through the use of internal calibrators that are coamplified with the sample nucleic acids and subsequently identified.

The technique is applicable to a variety of biological sources including, retroviruses, replicating virus bacteria and disease states such as cancer. Commercial kits for performing NASBA assays are sold by Life sciences, Inc., 2900 72nd St. North, St. Petersburg, Fla. 33710-4323.

Loop-mediated isothermal amplification or “LAMP” is a highly specific method of amplifying nucleic acids under isothermal conditions. The technique uses specific primers, such as those described herein, which recognize distinct sequences within the nucleic acid to be amplified. The reaction is initiated using a inner sequence primer containing sequences of the sense and antisense strands of the target nucleic acid. Following strand displacement nucleic acid synthesis primed by an outer primer releases a single-stranded nucleic acid. The single-stranded nucleic acid then serves as template for synthesis primed by the second inner and outer primers that hybridize to the other end of the target, producing a stem-loop structure. In subsequent LAMP cycling, one inner primer hybridizes to the loop on the product and initiates displacement synthesis, yielding the original stem-loop and a new stem-loop with a stem twice as long as that of the original structure.

The final products LAMP amplification are stem-loop structures with several inverted repeats of the target and cauliflower-like structures with multiple loops formed by annealing between alternately inverted repeats of the target in the same strand. For a complete explanation of LAMP amplification, See Notomi et al., Nucleic Acids Res. (2000) 28 (12):e63.

Primers and probes of the present invention also find utility in branched DNA signal ampliphication techniques. These techniques may use novel nucleotides such as isoC and isoG to prevent non-specific hybridization. By controlling non-specific hybridization signal amplification is increased. For a detailed description of the technique, see Collins et al., Nucleic Acids Res. (1997) 25 (15):2979-2984.

VI. Kits

The present invention also includes kits that aid in the use of the primers, probes and methods described herein. For example, one embodiment is a kit that includes a primer set of the present invention bundled with instructions for their use. Typically such instructions include a detailed description of a method for PCR analysis of a SARS-CoV target nucleic acid, as described herein. Kits of the present invention may optionally comprise a probe suitable for use with the primer set provided. Some embodiments also include standard target nucleic acids that are constructed for use with the provided primer pair.

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

As can be appreciated from the disclosure provided above, the present invention has a wide variety of applications. Accordingly, the following examples are offered for illustration purposes and are not intended to be construed as a limitation on the invention in any way. Those of skill in the art will readily recognize a variety of noncritical parameters that could be changed or modified to yield essentially similar results.

### EXAMPLES

### Example 1

### Detecting Plasma-Borne SARS Nucleic Acids and Prognostic Predictions Based on SARS Nucleic Acid Levels

To determine the detectability of SARS-CoV in plasma/serum, samples from 10 confirmed SARS patients were subjected to an optimized RNA extraction protocol and tested to determine if SARS-CoV RNA was detectable using the real-time RT-PCR assay, described below. Our results demonstrate that the plasma RNA assay is able to detect SARS-CoV in 80% (8/10) of the SARS patients.

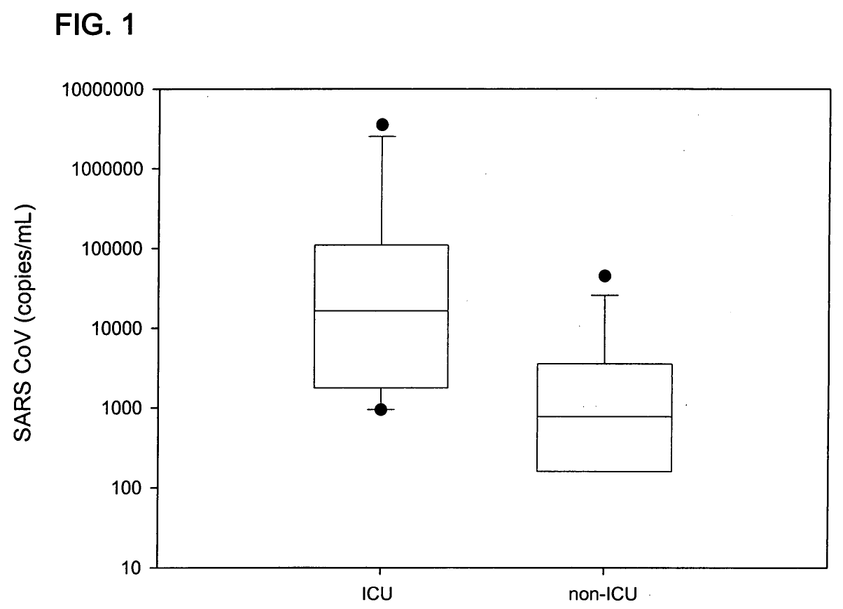

In this study, patients may be categorized into two prognostic groups: (a) the poor prognostic group consists of 12 patients who required admission to the intensive care unit (ICU); and (b) the good prognostic group consists of 16 patients who did not require ICU admission. The mean ages of group (a) and (b) were 58 and 46, respectively. There was no significant different between these two groups (t-test, p=0.06). Our data showed that the detection rates of group (a) and (b) were 100% (12/12) and 81% (13/16), respectively. The median levels of virus in group (a) and group (b) were 16360 and 772 copies/mL, respectively (FIG. 1). The median viral load of group (a) was 21-fold higher than that of group (b) and this difference was statistically significant (Mann-Whitney test, p=0.002).

If SARS-CoV is released from cells damaged through the pathological processes involved in SARS, the amount of SARS-CoV in plasma/serum might have prognostic implication in SARS patients. We hypothesized that SARS patients with a higher amount of virus on admission are of a poorer prognosis than those with a lower admission viral loads. To test this hypothesis, archived admission serum samples from 28 SARS patients from the Prince of Wales Hospital with informed consent were retrieved and the levels of SARS-CoV RNA in serum were measured.

Material & Methods

RNA Extraction.

Viral RNA was extracted from 0.28 mL of serum by QIAamp viral RNA mini kit (Qiagen, Hilden, Germany) according to the manufacturer's recommendations. RNA was then eluted with 50 μL of buffer and stored at −80° C.

Real-Time Quantitative RT-PCR.

One-step real-time quantitative RT-PCR was used for SARS-CoV RNA quantitation. Based on our public released complete genomic sequences of the virus (GenBank accession number AY278554 and AY282752), several pairs of primer specifically amplify SARS-CoV genome were designed. The sequences of primer and dual-labeled fluorescent probe were:

| | |
|---|---|
| SEQ ID NO:1 | |
| SARS1-F | 5′-GAGTGTGCGCAAGTATTAAGTGA-3′ |
| | (forward) |
| SEQ ID NO:2 | |
| SARS1-R | 5′-TGATGTTCCACCTGGTTTAACA-3′ |
| | (reverse) |
| SEQ ID NO:3 | |
| SARS1-P | 5′-FAM-ATGGTCATGTGTGGCGGCTCACTA- |
| | TAMRA-3′ (probe) |
| SEQ ID NO:4 | |
| SARS2-F | 5′-CCGCGAAGAAGCTATTCGT-3′ (forward) |
| SEQ ID NO:5 | |
| SARS2-R | 5′-TGCATGACAGCCCTCTACAT-3′ (reverse) |
| SEQ ID NO:6 | |
| SARS2-P | 5′-FAM-CGTTCGTGCGTGGATTGGCTT-TAMRA-3′ |
| | (probe) |
| SEQ ID NO:7 | |
| SARS3-F | 5′-TGCCCTCGCGCTATTG-3′ (forward) |
| SEQ ID NO:8 | |
| SARS3-R | 5′-GGCCTTTACCAGAAACTTTGC-3′ (reverse) |
| SEQ ID NO:9 | |
| SARS3-P | 5′-FAM-TGCTAGACAGATTGAACCAGCTTG-TAMRA- |
| | 3′ (probe) |
| SEQ ID NO:10 | |
| SARS4-F | 5′-CCGCTCATGGAAAGTGAACT-3′ (forward) |
| SEQ ID NO:11 | |
| SARS4-R | 5′-CGGCCATTCGCAAGTG-3′ (reverse) |
| SEQ ID NO:12 | |
| SARS4-P | 5′-FAM-TCATTGGTGCTGTGATCATTCGTGG- |
| | TAMRA-3′ (probe) |

Calibration curves for SARS-CoV RNA quantifications were prepared by serial dilutions of high performance liquid chromatography (HPLC)-purified single stranded synthetic DNA oligonucleotides (PROLIGO, Singapore) specifying the corresponding amplicons with concentrations ranging from 1×10⁷ copies to 5×10¹⁰ copies. Those assays were able to detect 5 copies of the respective calibrator target. Absolute concentration of SARS-CoV was expressed as copies/mL of plasma. The sequences of the synthetic calibrator were:

| | |
|---|---|
| SEQ ID NO:13 | |
| SARS1-std: | 5′-AACGAGTGTGCGCAAGTATTAAGTGAGATGGTCATGTGTGGCGGCTCACTA |
| | TATGTTAAACCAGGTGGAACATCATCCGG-3′ |
| SEQ ID NO:14 | |
| SARS2-std: | 5′-TCACCCGCGAAGAAGCTATTCGTCACGTTCGTGCGTGGATTGGCTTTGATGT |
| | AGAGGGCTGTCATGCAACTA-3′ |
| SEQ ID NO:15 | |
| SARS3-std: | 5′-GAAACTGCCCTCGCGCTATTGCTGCTAGACAGATTGAACCAGCTTGAGAGC |
| | AAAGTTTCTGGTAAAGGCCAACAA-3′ |
| SEQ ID NO:16 | |
| SARS4-std: | 5′-ACCAGACCGCTCATGGAAAGTGAACTTGTCATTGGTGCTGTGATCATTCGTG |
| | GTCACTTGCGAATGGCCGGACACT-3′ |

The RT-PCR reactions were set up according to the manufacturer's instructions (EZ rTth RNA PCR reagent set, Applied Biosystems, Foster City, Calif.) in a reaction volume of 25 μL. The primers and fluorescent probe were used at concentrations of 300 nM and 100 nM, respectively. 12 μL of extracted plasma RNA was used for amplification. Each sample was analyzed in duplicate, and the calibration curve was run in parallel for each analysis. Multiple negative water blanks were also included in every analysis.

### Example 2

### Detection of SARS Nucleic Acids in Pediatric Patients

Pediatric SARS patients have been reported to have a milder course of disease than adults. We investigated if SARS-CoV RNA can be detected in the plasma of pediatric patients during different stages of SARS and to study the correlation between viral loads and therapeutic treatment.

### Patients and Methods

Subjects

Peripheral blood samples were collected from all confirmed pediatric SARS patients admitted to the New Territories East Cluster of Hospital Authority Hospitals in Hong Kong. Samples were recruited between 13 Mar. 2003 and 17 May 2003. Serial blood samples were obtained from 8 pediatric SARS patients, starting from the day of hospital admission. The samples were obtained during routine blood tests for monitoring lymphocyte counts and biochemical parameters and enzymes. Informed consent was obtained from the patients or their parents and ethics approval was obtained from the institutional review board. As negative controls, blood samples from 15 pediatric patients who suffered from fever and infections other than SARS were collected. For comparison with the pediatric viral load data, plasma samples from 13 adult SARS patients taken within the first week of fever onset were also collected.

Processing of Blood Samples and RNA Extraction

Blood samples were collected in EDTA-containing tubes, and centrifuged at 1600×g for 10 min at 4° C. Plasma was then carefully transferred into plain polypropylene tubes. RNA extraction was performed as described in Example 1.

Real-Time Quantitative RT-PCR

One-step real-time quantitative RT-PCR was used for SARS-CoV RNA quantification, a RT-PCR system specifically targeting the polymerase gene (orf1 ab polyprotein: 15327-15398 nt, Accession no. AY278554, (Tsui SK, et al., N Engl J Med 2003;349:187-8)) of the SARS-CoV genome was designed as described in Example 1. The primer sequences were:

| | |
|---|---|
| SEQ ID NO:1 | 5′-GAGTGTGCGCAAGTATTAAGTGA-3′ (forward) |
| and | |
| SEQ ID NO:2 | 5′-TGATGTTCCACCTGGTTTAACA-3′ (reverse) |

The dual-labeled fluorescent probe was:
SEQ ID NO:3
   5′-(FAM)ATGGTCATGTGTGGCGGCTCACTA(TAMRA)-3′

A calibration curve for SARS-CoV RNA quantification was prepared by serial dilutions of a high performance liquid chromatography-purified single stranded synthetic DNA oligonucleotide (PROLIGO, Singapore), with concentrations ranging from 1×10¹ copies to 1×10⁷ copies. Concentrations of SARS-CoV were expressed as copies/mL of plasma. The sequence of the synthetic DNA oligonucleotide for calibration purposes was previously described in Example 1.

The RT-PCR reactions were set up according to the manufacturer's instructions (EZ rTth RNA PCR reagent set, Applied Biosystems, Foster City, Calif.) in a reaction volume of 25 μL. The primers and fluorescent probe were used at concentrations of 300 nM and 100 nM, respectively. 12 μL of extracted plasma RNA was used for amplification. The thermal profile used for the analysis was as follows: the reaction was initiated at 50° C. for 2 min for the included uracil N-glycosylase to act, followed by reverse transcription at 60° C. for 30 min. After a 5-min denaturation at 95° C., 40 cycles of PCR was carried out using denaturation at 94° C. for 20 s and 1 min annealing/extension at 56° C. Each sample was analyzed in duplicate, and the calibration curve was run in parallel for each analysis. Multiple negative water blanks were also included in every analysis.

Statistical Analysis

Statistical analysis was performed using the Sigma Stat 2.03 software (SPSS).

Detectability of Plasma SARS-CoV RNA from Pediatric Patients

To investigate if SARS-CoV RNA could be detected in the plasma of pediatric patients, 8 confirmed cases were studied. The median age of this cohort was 10.3 years (range: 0.3 to 17.5 years). Plasma samples were taken within the first week of hospital admission, representing a mean of 5 days after fever onset (range: 3 to 7 days). One subject did not have IgG seroconversion during his illness. Plasma SARS-CoV RNA was detected in 7 out of the 8 pediatric patients (87.5%), including the subject who did not have IgG seroconversion. The median plasma SARS-CoV RNA concentration was 357 copies/mL (interquartile range: 182 to 529 copies/mL). As negative controls, SARS-CoV RNA was not detected in the plasma samples obtained from 15 pediatric patients who suffered from non-SARS-related infections.

Serial Analysis of SARS-CoV RNA in Plasma of Pediatric Patients

To study the relative usefulness of plasma SARS-CoV measurement at different stages of the disease, serial plasma samples were collected from these 8 pediatric SARS patients and were subjected to SARS-CoV measurement. At day 7 after fever onset, plasma SARS-CoV RNA was detected in all patients (100%). The median plasma SARS-CoV RNA concentration was 483 copies/mL (interquartile range: 338 to 1237 copies/mL). The detection rate dropped to 62.5% (5 of 8) at day 14 after fever onset. The median plasma SARS-CoV RNA concentration at day 14 was 103 copies/mL (interquartile range: 0 to 957 copies/mL).

Plasma SARS-CoV Viral Load Comparison Between Adult and Pediatric Patients

To examine whether the plasma SARS-CoV viral load of pediatric SARS patients is different from that of adult SARS patients, the pediatric data were compared with the data of 13 adult SARS patients. The adult plasma samples were taken within the first week of fever onset. No significant difference was observed between the plasma SARS-CoV RNA concentration in pediatric and adult SARS patients taken within the first week of hospital admission (Mann-Whitney test, P=0.096). In addition, we compared the plasma SARS-CoV RNA concentration at day 7 after fever onset, and once again, no significant difference was observed between pediatric and adult SARS patients (Mann-Whitney test, P=0.076).

Drug Treatments and Viral Loads

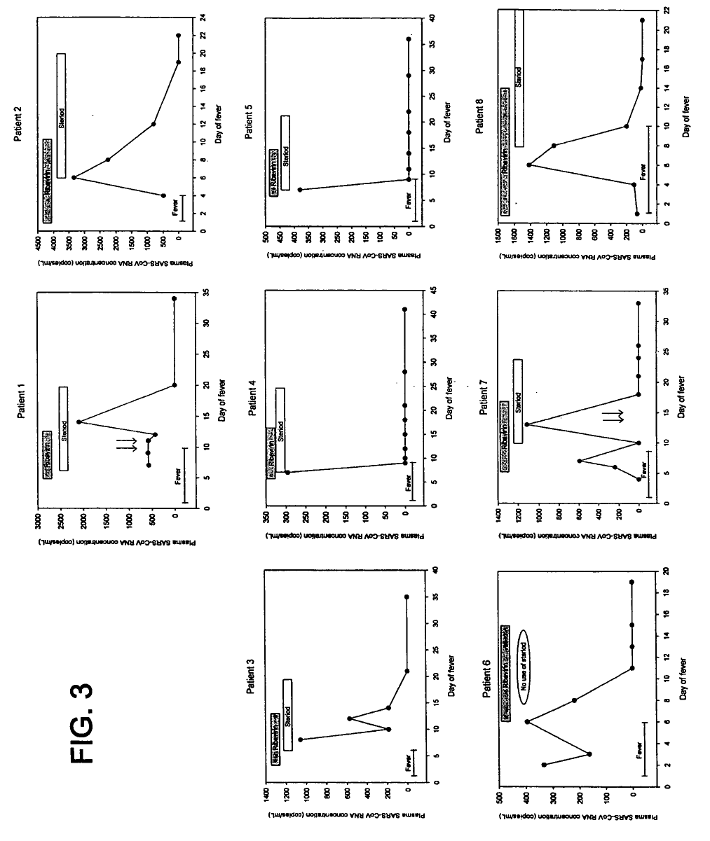

All 8 studied subjects satisfied the WHO surveillance case definition for SARS (Hon K L, et al., Lancet 2003;361:1701-3). Seven of them had been in close contact with infected adults except patient 7 who had no SARS contact history. All patients had fever and the mean duration of fever was 8 days (range: 4 to 10 days). During the course of hospitalization, all patients were initially treated with oral ribavirin (40-60 mg/kg daily) (FIG. 3). Treatment continued for a mean duration of 10 days (range: 3 to 14 days). All patients, except patient 6, were treated with oral prednisolone starting at a mean of 7 days (range: 6 to 10 days) after fever onset and the duration of prednisolone treatment was 14 days. For patients 1 and 7, pulse intravenous methylprednisolone was used (10-20 mg/kg).

Serial plasma viral load analysis showed that the concentration of SARS-CoV RNA in the plasma of the 8 pediatric patients peaked at a mean of 8 days after fever onset (range: 6 to 13 days). Plasma SARS-CoV RNA concentration dropped to zero after day 21 after fever onset (FIG. 3).

A Serologically Negative Patient with Detectable SARS-CoV RNA in Plasma

Among the 8 studied subjects, patient 8 (4-month-old) remained serologically negative for antibodies against SARS-CoV, despite having detectable SARS-CoV RNA in his plasma samples. One throat swab sample was also found to be positive for SARS-CoV RNA by RT-PCR. Four other family members, including his parents, were diagnosed with SARS prior to the onset of fever in patient 8. The concentration of SARS-CoV RNA in plasma peaked at 8 days after fever onset and was still detectable up to 14 days after fever onset. SARS-CoV RNA became undetectable in the plasma on day 17 after fever onset.

### Discussion

This example demonstrates that SARS-CoV RNA is detectable in the plasma of pediatric patients with a detection rate of 87.5% within the first week of hospital admission, and detection rates of 100% at day 7 and 62.5% at day 14 after fever onset. These data are largely concordant with our previous data in adult SARS patients showing a 75%-78% detection rate for plasma/serum SARS-CoV RNA within the first week of illness (Example 1). Taken together, these data suggest that plasma SARS-CoV measurement is a sensitive method for detecting SARS-CoV infection during the first week after fever onset.

The serial data presented here have demonstrated that the concentration of SARS-CoV RNA in plasma from the studied patients peaked at a mean of 8 days after fever onset (range: 6 to 13 days). Four of the pediatric cases (50%) still had detectable SARS-CoV RNA in plasma up to 15 days after fever onset. We did not observe any systemic correlation between the plasma viral load and steroid or ribavirin treatment.

Recent studies have reported that the clinical course of pediatric SARS patients was less severe in comparison with adult SARS patients (Chiu W K, et al., Pediatr Crit Care Med 2003;4:279-83; and Hon K L, et al., Lancet 2003;361:1701-3). No significant differences in plasma SARS-CoV viral load were observed between pediatric and adult SARS patients (Example 1) taken within the first week of admission and at day 7 after fever onset. Thus, investigation on other clinical parameters such as lymphocyte counts would be required so as to explain the relatively milder clinical course of pediatric SARS patients.

An interesting finding was that patient 8, a 4-month-old infant who had been in close contact with other infected members of his family, remained serologically negative for antibodies against SARS-CoV despite the presence of SARS-CoV in his plasma. In serological testing for SARS, adult patients had been reported to have a sensitivity of 93% at day 28 after symptom onset (Peiris J S, et al., Lancet 2003;361:1767-72).

In conclusion, viremia appears to be a consistent feature in both pediatric and adult SARS patients. The relatively high detection of SARS-CoV in plasma and serum during the first week of illness suggests that plasma/serum-based RT-PCR should be incorporated into the routine diagnostic workup of suspected or confirmed SARS patients both in adult and pediatric populations.

### Acknowledgements

This work is supported by the Hong Kong Research Grants Council Special Grants for SARS Research (CUHK 4508/03M).

### Example 3

### Early Detection of SARS-CoV

The availability of an early diagnostic tool for severe acute respiratory syndrome (SARS) would be of major public health implication. We investigated if the SARS coronavirus (SARS-CoV) can be detected in serum and plasma samples during the early stage of SARS and to study the potential prognostic implications of such an approach.

### Patients and Methods

Subjects

Peripheral blood samples were obtained from SARS patients admitted to the New Territories East Cluster of Hospital Authority Hospitals in Hong Kong. Samples were recruited between March 2003 and May 2003.

In the first part of this study, blood samples were collected from 12 SARS patients on the day of hospital admission, as well as at day 7 and day 14 after fever onset. Informed consent was obtained from the patients and ethics approval was obtained from the institutional review board. In the second part of this study, blood samples were obtained from 23 SARS patients on the day of hospital admission. All studied subjects had subsequent serological evidence of antibodies towards SARS-CoV. For the prognostic part of the study, the previously mentioned 23 SARS patients were subdivided into two patient groups: (a) 11 patients who required admission to the Intensive Care Unit (ICU) and (b) 12 patients who did not require ICU admission (non-ICU), during the duration of their hospitalization.

Processing of Blood Samples and RNA Extraction

Blood samples were collected in EDTA-containing tubes or plain tubes, and centrifuged at 1600×g for 10 min at 4° C. Plasma or serum was then carefully transferred into plain polypropylene tubes. The plasma samples were re-centrifuged at 16000×g for 10 min at 4° C., and the supernatants were collected into fresh polypropylene tubes. RNA extraction was performed as described in example 1.

Real-Time Quantitative RT-PCR

One-step real-time quantitative RT-PCR was used for SARS-CoV RNA quantitation. Based on the publicly released full genomic sequences of SARS-CoV (http://www.ncbi.nlm.nih.gov), two RT-PCR systems specifically targeting the SARS-CoV genome were designed. The SARSPol1 system targeted the polymerase gene (orf1ab polyprotein: 15327-15398 nt, Accession no. AY278554) and the SARSN system targeted the nucleocapsid gene (N: 28758-28823 nt, Accession no. AY278554) of the SARS-CoV genome. The SARSPol1 primer sequences were:

| | |
|---|---|
| SEQ ID NO:1 | 5′-GAGTGTGCGCAAGTATTAAGTGA-3′ (forward) |
| and | |
| SEQ ID NO:2 | 5′-TGATGTTCCACCTGGTTTAACA-3′ (reverse). |

The dual-labeled fluorescent probe was:

| | |
|---|---|
| SEQ ID NO:3 | 5′-(FAM)ATGGTCATGTGTGGCGGCTCACTA |
| | (TAMRA)-3′. |

The SARSN primer sequences were:

| | |
|---|---|
| SEQ ID NO:7 | 5′-TGCCCTCGCGCTATTG-3′ (forward) and |
| SEQ ID NO:8 | 5′-GGCCTTTACCAGAAACTTTGC-3′ (reverse). |

The dual-labeled fluorescent probe was:

| | |
|---|---|
| SEQ ID NO:9 | 5′-(FAM)TGCTAGACAGATTGAACCAGCTTG |
| | (TAMRA)-3′. |

Calibration curves for SARS-CoV RNA quantification were prepared by serial dilutions of a high performance liquid chromatography-purified single stranded synthetic DNA oligonucleotide (PROLIGO, Singapore), with concentrations ranging from 1×10¹ copies to 1×10⁷ copies. The assay was able to detect 10 copies of the calibrator target. Concentrations of SARS-CoV were expressed as copies/mL of plasma/serum. The sequences of the synthetic DNA oligonucleotides for SARSPol1 and SARSN calibrations were:

| | |
|---|---|
| SEQ ID NO:13 | 5′-AACGAGTGTGCGCAAGTATTAAGTGAGATGGTCATGTGTGGCGGCTC |
| ACTATATGTTAAACCAGGTGGAACATCATCCGG-3′ and | |
| SEQ ID NO:15 | 5′-GAAACTGCCCTCGCGCTATTGCTGCTAGACAGATTGAACCAGCTTGAGAGC |
| AAAGTTTCTGGTAAAGGCCAACAA-3′, respectively. | |

The RT-PCR reactions were set up according to the manufacturer's instructions (EZ rTth RNA PCR reagent set, Applied Biosystems, Foster City, Calif.) in a reaction volume of 25 μL. The primers and fluorescent probes were used at concentrations of 300 nM and 100 nM, respectively. 12 μL of extracted plasma/serum RNA was used for amplification. The thermal profile used for the analysis was as follows: the reaction was initiated at 50° C. for 2 min for the included uracil N-glycosylase to act, followed by reverse transcription at 60° C. for 30 min. After a 5-min denaturation at 95° C., 40 cycles of PCR was carried out using denaturation at 94° C. for 20 s and 1 min annealing/extension at 56° C. Each sample was analysed in duplicate, and the calibration curve was run in parallel for each analysis. Multiple negative water blanks were also included in every analysis.

Statistical Analysis

Statistical analysis was performed using the Sigma Stat 2.03 software (SPSS). Student t-test was used for the comparison of the ages of ICU and non-ICU groups. Mann-Whitney test was used for the comparison of serum SARS-CoV RNA concentrations between ICU and non-ICU groups. Pearson correlation analysis was used to analyze the correlation of SARS-CoV RNA concentrations between the SARSPol1 and SARSN RT-PCR systems.

### Results

Development of Real-Time Quantitative RT-PCR

To determine the quantitative performance of the SARS-CoV RT-PCR assays, these assays were used to amplify serially diluted calibrators which were synthetic DNA oligonucleotides based on the SARS-CoV genomic sequence. The calibration curves for the SARSPol1 (polymerase) and SARSN (nucleocapsid) amplification systems demonstrated a dynamic range from 1×10¹ to 1×10⁷ copies. A semi-logarithmic plot of different calibrator concentrations against the threshold cycles yielded correlation coefficients of 0.987 for the SARSPol1 system and 0.993 for the SARSN system. The sensitivities of the amplification steps of these assays were sufficient to detect 10 copies of the SARSPol 1 target and 5 copies of the SARSN target. To determine the precision of the whole analytical procedure involving RNA extraction, reverse transcription and amplification steps, we performed 10 replicate RNA extractions from a sample pooled from plasma obtained from 5 SARS patients and subjected these extracted RNA samples to RT-PCR assays. The coefficients of variation of the copy number of these replicate analyses for the SARSPol1 and SARSN amplification systems were 16.4% and 14.9%, respectively.

Detectability of Plasma SARS-CoV RNA at Different Stages of the Disease

To investigate if SARS-CoV RNA could be detected in plasma and to study the relative usefulness of plasma SARS-CoV measurement at different stages of the disease, 12 serologically confirmed SARS patients were studied. Plasma samples were taken on admission, representing a mean of 3.6 days after fever onset (range: 1 to 6 days). Further samples were taken from each of these subjects at day 7 and day 14 after fever onset. Using the SARSPol1 real-time RT-PCR system, plasma SARS-CoV RNA was detected in 9 patients on admission (75%). The detection rate remained at 75% (9 of 12) at day 7 and fell to 42% (5 of 12) at day 14 after fever onset. As negative controls, SARS-CoV RNA was not detected in the plasma samples obtained from 40 healthy individuals.

Quantitative Analysis of SARS-CoV RNA in Sera of SARS Patients

To test the detectability of SARS-CoV RNA in the serum, instead of plasma, during the early stage of SARS, 23 serum samples obtained from SARS patients on the day of hospital admission were analyzed using the SARSPol1 real-time RT-PCR system. For 22 subjects, the serum samples were taken at a mean of 2.6 days (range: 1 to 6 days) following the onset of fever. One subject did not have fever during his illness. SARSPol1 system was able to detect SARS-CoV RNA in 18 of the 23 samples (78%), including the subject who did not have fever. The detection rate essentially confirmed the plasma-based results from our first cohort as described above. The median serum SARS-CoV RNA concentration was 752 copies/mL (interquartile range: 54 to 5728 copies/mL). As negative controls, SARS-CoV RNA was not detected in serum samples obtained from 30 healthy individuals.

Corroborative Data from a Second RT-PCR System

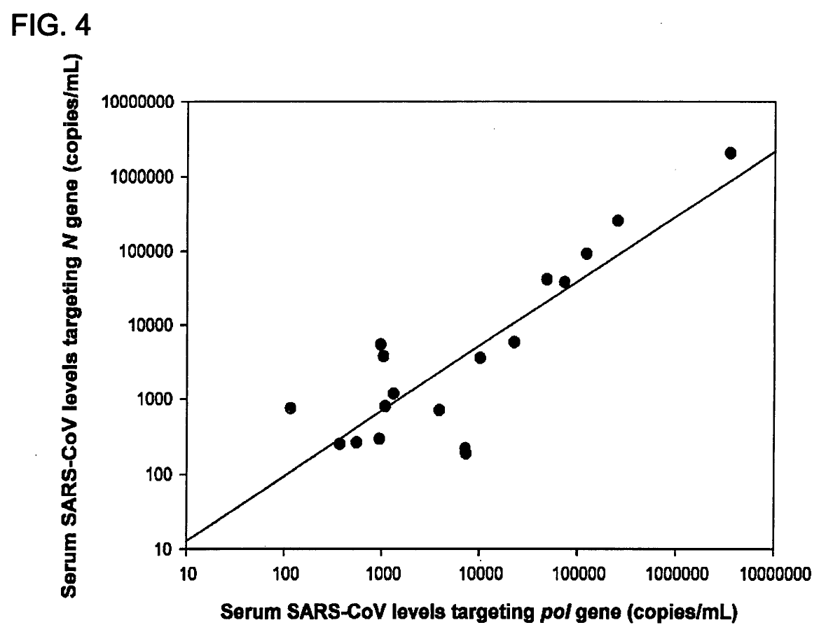

To confirm the data generated by the SARSPol1 RT-PCR system, another real-time RT-PCR system (SARSN) targeting the nucleocapsid gene of the SARS-CoV genome was used to repeat the serum analysis. The SARSN system was able to detect SARS-CoV RNA in 20 of the 23 samples (87%). The median serum SARS RNA concentration was 3876 copies/mL (interquartile range: 763 to 39112 copies/mL). The correlation coefficient of SARS-CoV RNA concentrations between the SARSPol1 and SARSN RT-PCR systems was 0.998 (Pearson correlation analysis, P<0.001, FIG. 4). As negative controls, SARS-CoV RNA was not detected by the SARSN RT-PCR system in serum samples obtained from 30 healthy individuals.

Prognostic Implication

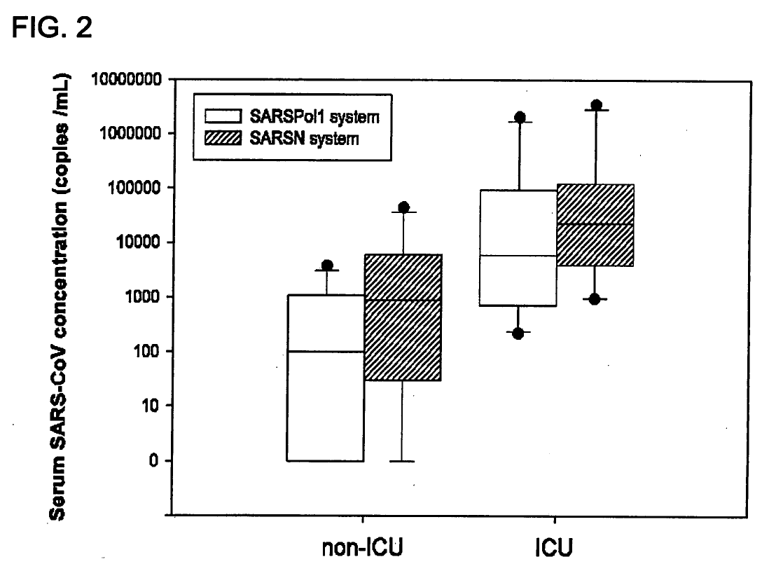

To determine the potential prognostic implication of the admission serum SARS-CoV concentration, the previously mentioned group of 23 patients were stratified into those who required admission to the ICU (N=11) and those who did not (N=12), during the duration of their hospitalisation. The ages between these groups showed no statistically significant difference (Student t-test, P=0.188). The male to female ratios were 5:6 for ICU group and 6:6 for non-ICU group. For the SARSPol1 RT-PCR assay, the median serum SARS-CoV concentrations in the ICU and non-ICU groups were 5828 copies/mL (interquartile range: 1422 to 79794 copies/mL) and 99 copies/mL (interquartile range: 0 to 969 copies/mL), respectively (FIG. 2). The serum SARS-CoV concentrations between these groups showed statistically significant difference (Mann-Whitney test, P<0.005). For the SARSN RT-PCR assay, the median serum SARS-CoV concentrations in the ICU and non-ICU groups were 22527 copies/mL (interquartile range: 4707 to 110135 copies/mL) and 868 copies/mL (interquartile range: 58 to 4487 copies/mL), respectively (FIG. 2). The serum SARS-CoV concentrations between these groups also showed statistically significant difference (Mann-Whitney test, P<0.007).

### Discussion

This example demonstrates that SARS-CoV RNA is detectable in the plasma and sera of patients during the early stage of SARS, with the detection rate of 75%-78% for the SARSPol1 system and 87% for the SARSN system. The findings demonstrate that plasma/serum SARS-CoV measurement is a sensitive method for detecting SARS-CoV infection during the first week after fever onset. This sensitivity is much higher than those reported for other clinical specimen types at a similar stage of infection. For example, RT-PCR analysis of nasopharyngeal aspirates had been reported to have a sensitivity of 32% in the first week after symptom onset (Peiris J S, et al., Lancet 2003;361:1767-72).

The data presented here have also demonstrated that the median concentrations of serum SARS-CoV RNA in patients who required ICU admission during the course of hospitalisation was 29.6 and 25.9 times higher than those who did not require intensive care by using the SARSPol1 and SARSN RT-PCR systems, respectively. Our results showed that there is a strong correlation in the serum SARS-CoV RNA concentrations obtained by the SARSPol1 and SARSN RT-PCR systems, though the median serum SARS-CoV concentrations differed. The differences observed in the quantitative levels may be a result of the differences in the amplification efficiency or the co-existence of sub-genomic fragments of the N-gene with the full virus genome in serum. This would be a subject of further investigation.

Plasma/serum SARS-CoV measurement can function in a synergistic manner with existing diagnostic strategies for SARS. Thus, plasma/serum RT-PCR can be performed with high sensitivity during the first week of the disease, RT-PCR analysis of stool and respiratory samples can be performed during the second week, and serological testing for antibodies against SARS-CoV can be used from day 21 onwards (Peiris J S, et al., Lancet 2003;361:1767-72). The availability of a diagnostic test for the early identification of SARS patients would be useful in the public health control of SARS. Furthermore, our data shows that serum SARS-CoV measurement is a prognostic marker which can be used even on the first day of hospital admission. Apart from its obvious clinical significance, this observation also suggests that a high systemic viral load may either directly result in more severe tissue damage or indirectly through the activation of a potentially damaging immune reaction.

SEQUENCE LISTING

| | |
|---|---|
| SEQ ID NO:1 | |
| SARS1-F | 5′-GAGTGTGCGCAAGTATTAAGTGA-3′ |
| | (forward) |
| SEQ ID NO:2 | |
| SARS1-R | 5′-TGATGTTCCACCTGGTTTAACA-3′ (reverse) |
| SEQ ID NO:3 | |
| SARS1-P | 5′-FAM-ATGGTCATGTGTGGCGGCTCACTA-TAMRA- |
| | 3′ (probe) |
| SEQ ID NO:4 | |
| SARS2-F | 5′-CCGCGAAGAAGCTATTCGT-3′ (forward) |
| SEQ ID NO:5 | |
| SARS2-R | 5′-TGCATGACAGCCCTCTACAT-3′ (reverse) |
| SEQ ID NO:6 | |
| SARS2-P | 5′-FAM-CGTTCGTGCGTGGATTGGCTT-TAMRA-3′ |
| | (probe) |
| SEQ ID NO:7 | |
| SARS3-F | 5′-TGCCCTCGCGCTATTG-3′ (forward) |
| SEQ ID NO:8 | |
| SARS3-R | 5′-GGCCTTTACCAGAAACTTTGC-3′ (reverse) |
| SEQ ID NO:9 | |
| SARS3-P | 5′-FAM-TGCTAGACAGATTGAACCAGCTTG-TAMRA- |
| | 3′ (probe) |
| SEQ ID NO:10 | |
| SARS4-F | 5′-CCGCTCATGGAAAGTGAACT-3′ (forward) |
| SEQ ID NO:11 | |
| SARS4-R | 5′-CGGCCATTCGCAAGTG-3′ (reverse) |
| SEQ ID NO:12 | |
| SARS4-P | 5′-FAM-TCATTGGTGCTGTGATCATTCGTGG-TAMRA- |
| | 3′ (probe) |
| SEQ ID NO:13 | |
| SARS1- | 5′-AACGAGTGTGCGCAAGTATTAAGTGAGATGGTCA |
| standard | TGTGTGGCGGCTCACTATATGTTAAACCAGGTGGAAC |
| target | ATCATCCGG-3′ |
| nucleic | |
| acid: | |
| SEQ ID NO:14 | |
| SARS2- | 5′-TCACCCGCGAAGAAGCTATTCGTCACGTTCGTGC |
| standard | GTGGATTGGCTTTGATGTAGAGGGCTGTCATGCAACT |
| target | A-3′ |
| nucleic | |
| acid: | |
| SEQ ID NO:15 | |
| SARS3- | 5′-GAAACTGCCCTCGCGCTATTGCTGCTAGACAGAT |
| standard | TGAACCAGCTTGAGAGCAAAGTTTCTGGTAAAGGCCA |
| target | ACAA-3′ |
| nucleic | |
| acid: | |
| SEQ ID NO:16 | |
| SARS4- | 5′-ACCAGACCGCTCATGGAAAGTGAACTTGTCATTG |
| standard | GTGCTGTGATCATTCGTGGTCACTTGCGAATGGCCGG |
| target | ACACT-3′ |
| nucleic | |
| acid: | |

1. A primer pair comprising:
(i) a first primer having at least 10 contiguous nucleotides being at least 75% identical to an oligonucleotide A, or fragment thereof, and hybridizing under stringent conditions to a reference oligonucleotide complementary to oligonucleotide A; and
(ii) a second primer having at least 10 contiguous nucleotides being at least 75% identical to an oligonucleotide B, or fragment thereof, and hybridizing under stringent conditions to a reference oligonucleotide complementary to oligonucleotide B;
wherein oligonucleotide A and oligonucleotide B are selected from the group consisting of SEQ ID NO:1 and 2, SEQ ID NO:4 and 5, SEQ ID NO:7 and SEQ ID NO:8, and SEQ ID NO:10 and 11.

2. The primer pair according to claim 1, wherein at least the first primer or the second primer comprises at least 16 nucleotides.

3. A method for detecting the presence of SARS-CoV in a sample comprising the steps of:
(a) contacting the sample with a primer pair comprising:
(i) a first primer having at least 10 contiguous nucleotides being at least 75% identical to an oligonucleotide A, or fragment thereof, and hybridizing under stringent conditions to a reference oligonucleotide complementary to oligonucleotide A; and
(ii) a second primer having at least 10 contiguous nucleotides being at least 75% identical to an oligonucleotide B, or fragment thereof, and hybridizing under stringent conditions to a reference oligonucleotide complementary to oligonucleotide B;
wherein oligonucleotide A and oligonucleotide B are selected from the group consisting of SEQ ID NO:1 and 2, SEQ ID NO:4 and 5, SEQ ID NO:7 and SEQ ID NO:8, and SEQ ID NO:10 and 11;
(b) performing RT-PCR on the sample, wherein, if present, a SARS-CoV-specific nucleic acid is amplified; and
(c) determining the presence or absence of SARS-CoV in the sample by respectively detecting or not detecting the SARS-CoV-specific nucleic acid.

4. A method of screening a population for the presence of SARS-CoV comprising analyzing a plurality of samples from the population by the method according to claim 3.

5. The method according to claim 3, wherein step (c) further comprises hybridizing a SARS-CoV-specific probe to the SARS-CoV-specific nucleic acid.

6. The method according to claim 5, wherein the SARS-CoV-specific probe comprises at least 10 contiguous nucleotides selected from the group of nucleotide sequences consisting of SEQ ID NO:3, SEQ ID NO:6, SEQ ID NO:9 and SEQ ID NO:12.

7. The method according to claim 3, wherein oligonucleotide A comprises the nucleotide sequence of SEQ ID NO:1 or a fragment of at least 10 contiguous nucleotides thereof; oligonucleotide B comprises the nucleotide sequence of SEQ ID NO:2 or a fragment of at least 10 contiguous nucleotides thereof; and step (c) further comprises hybridizing at least 10 contiguous nucleotides of SEQ ID NO:3 to the SARS-CoV-specific nucleic acid.

8. The method according to claim 3, wherein oligonucleotide A comprises the nucleotide sequence of SEQ ID NO:4 or a fragment of at least 10 contiguous nucleotides thereof;
oligonucleotide B comprises the nucleotide sequence of SEQ ID NO:5 or a fragment of at least 10 contiguous nucleotides thereof; and step (c) further comprises hybridizing at least 10 contiguous nucleotides of SEQ ID NO:6 to the SARS-CoV-specific nucleic acid.

9. The method according to claim 3, wherein oligonucleotide A comprises the nucleotide sequence of SEQ ID NO:7 or a fragment of at least 10 contiguous nucleotides thereof; oligonucleotide B comprises the nucleotide sequence of SEQ ID NO:8 or a fragment of at least 10 contiguous nucleotides thereof; and step (c) further comprises hybridizing at least 10 contiguous nucleotides of SEQ ID NO:9 to the SARS-CoV-specific nucleic acid.

10. The method according to claim 3, wherein oligonucleotide A comprises the nucleotide sequence of SEQ ID NO:10 or a fragment of at least 10 contiguous nucleotides thereof; oligonucleotide B comprises the nucleotide sequence of SEQ ID NO:11 or a fragment of at least 10 contiguous nucleotides thereof; and step (c) further comprises hybridizing at least 10 contiguous nucleotides of SEQ ID NO:12 to the SARS-CoV-specific nucleic acid.

11. A method of performing prognostic testing on an individual infected with SARS-CoV comprising the steps of:
(a) contacting a sample from the individual with a primer pair comprising:
(i) a first primer having at least 10 contiguous nucleotides being at least 75% identical to an oligonucleotide A, or fragment thereof, and hybridizing under stringent conditions to a reference oligonucleotide complementary to oligonucleotide A; and
(ii) a second primer having at least 10 contiguous nucleotides being at least 75% identical to an oligonucleotide B, or fragment thereof, and hybridizing under stringent conditions to a reference oligonucleotide complementary to oligonucleotide B;
wherein oligonucleotide A and oligonucleotide B are selected from the group consisting of SEQ ID NO:1 and 2, SEQ ID NO:4 and 5, SEQ ID NO:7 and SEQ ID NO:8, and SEQ ID NO:10 and 11;
(b) performing a RT-PCR on the sample, wherein, if present, a SARS-CoV-specific target nucleic acid is amplified;
(c) determining a SARS-CoV concentration by quantifying the amplification of the SARS-CoV-specific target nucleic acid and comparing the quantity with a standard calibration curve; and
(d) providing a prognosis based on the SARS-CoV concentration.

12. A method of screening a population for the presence of SARS-CoV comprising analyzing a plurality of samples from the population by the method according to claim 11.

13. The method according to claim 11, wherein the sample is a plasma sample formed by allowing clotting of a blood sample from the individual.

14. The method according to claim 11, wherein the standard calibration curve is constructed using one or more isolated single-stranded nucleic acids selected from the group consisting of SEQ ID NO:13, SEQ ID NO:14, SEQ ID NO:15 and SEQ ID NO:16.

15. The method according to claim 11, wherein oligonucleotide A comprises the nucleotide sequence of SEQ ID NO:1 or a fragment of at least 10 contiguous nucleotides thereof; oligonucleotide B comprises the nucleotide sequence of SEQ ID NO:2 or a fragment of at least 10 contiguous nucleotides thereof; and step (c) comprises hybridizing a probe having at least 10 contiguous nucleotides from SEQ ID NO:3 to SARS-CoV-specific nucleic acids in the sample to form a plurality of duplex molecules, quantifying the duplex molecules formed and determining SARS-CoV concentration by comparing the quantity of duplex molecules with a standard calibration curve.

16. The method according to claim 11, wherein oligonucleotide A comprises the nucleotide sequence of SEQ ID NO:4 or a fragment of at least 10 contiguous nucleotides thereof; oligonucleotide B comprises the nucleotide sequence of SEQ ID NO:5 or a fragment of at least 10 contiguous nucleotides thereof; and step (c) further comprises hybridizing a probe having at least 10 contiguous nucleotides from SEQ ID NO:6 to SARS-CoV-specific nucleic acids in the sample to form a plurality of duplex molecules, quantifying the duplex molecules formed and determining a SARS-CoV concentration by comparing the quantity of duplex molecules with a standard calibration curve.

17. The method according to claim 11, wherein oligonucleotide A comprises the nucleotide sequence of SEQ ID NO:7 or a fragment of at least 10 contiguous nucleotides thereof; oligonucleotide B comprises the nucleotide sequence of SEQ ID NO:8 or a fragment of at least 10 contiguous nucleotides thereof; and step (c) further comprises hybridizing a probe having at least 10 contiguous nucleotides from SEQ ID NO:9 to SARS-CoV-specific nucleic acids in the sample to form a plurality of duplex molecules, quantifying the duplex molecules formed and determining a SARS-CoV concentration by comparing the quantity of duplex molecules with a standard calibration curve.

18. The method according to claim 11, wherein oligonucleotide A comprises the nucleotide sequence of SEQ ID NO:10 or a fragment of at least 10 contiguous nucleotides thereof; oligonucleotide B comprises the nucleotide sequence of SEQ ID NO:11 or a fragment of at least 10 contiguous nucleotides thereof; and step (c) further comprises hybridizing a probe having at least 10 contiguous nucleotides from SEQ ID NO:12 to SARS-CoV-specific nucleic acids in the sample to form a plurality of duplex molecules, quantifying the duplex molecules formed and determining a SARS-CoV concentration by comparing the quantity of duplex molecules with a standard calibration curve.

19. A kit for the detection of the presence of SARS-CoV in a sample comprising:
(a) a primer pair comprising:
(i) a first primer having at least 10 contiguous nucleotides being at least 75% identical to an oligonucleotide A, or fragment thereof, and hybridizing under stringent conditions to a reference oligonucleotide complementary to oligonucleotide A; and
(ii) a second primer having at least 10 contiguous nucleotides being at least 75% identical to an oligonucleotide B, or fragment thereof, and hybridizing under stringent conditions to a reference oligonucleotide complementary to oligonucleotide B;
wherein oligonucleotide A and oligonucleotide B are selected from the group consisting of SEQ ID NO:1 and 2, SEQ ID NO:4 and 5, SEQ ID NO:7 and SEQ ID NO:8;, and SEQ ID NO:10 and 11; and
(b) instructions for using the primer pair.

20. The kit according to claim 19, further comprising a probe that specifically hybridizes to a SARS-CoV-specific nucleic acid, the SARS-CoV-specific nucleic acid specifically hybridizing to the first primer or the second primer.

21. The kit according to claim 19, further comprising one or more standard target nucleic acids selected from the group consisting of SEQ ID NO:13, SEQ ID NO:14, SEQ ID NO:15 and SEQ ID NO:16.

22. The kit according to claim 20, wherein oligonucleotide A comprises the nucleotide sequence of SEQ ID NO:1 or a fragment thereof; oligonucleotide B comprises the nucleotide sequence of SEQ ID NO:2 or a fragment thereof; and the probe comprises the nucleotide sequence of SEQ ID NO:3.

23. The kit according to claim 20, wherein oligonucleotide A comprises the nucleotide sequence of SEQ ID NO:4 or a fragment thereof; oligonucleotide B comprises the nucleotide sequence of SEQ ID NO:5 or a fragment thereof; and the probe comprises the nucleotide sequence of SEQ ID NO:6.

24. The kit according to claim 20, wherein oligonucleotide A comprises the nucleotide sequence of SEQ ID NO:7 or a fragment thereof; oligonucleotide B comprises the nucleotide sequence of SEQ ID NO:8 or a fragment thereof; and the probe comprises the nucleotide sequence of SEQ ID NO:9.

25. The kit according to claim 20, wherein oligonucleotide A comprises the nucleotide sequence of SEQ ID NO:10 or a fragment thereof; oligonucleotide B comprises the nucleotide sequence of SEQ ID NO:11 or a fragment thereof; and the probe comprises the nucleotide sequence of SEQ ID NO:12

26. An isolated nucleic acid at least 10 bases in length and comprising a nucleotide sequence at least 85% complementary to 10 or more contiguous nucleotides of a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 and SEQ ID NO:12 or fragments thereof, wherein the isolated nucleic acid hybridizes to a SARS-CoV-specific nucleic acid, but not nucleic acids specific to other coronaviruses under selectively stringent conditions.

27. The nucleic acid according to claim 26, wherein the nucleotide sequence is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11 and SEQ ID NO:12.


SEQUENCE LISTING (16 sequences)

SEQ ID NO: 1 - DNA, 23 bp, Artificial Sequence
  Description of Artificial Sequenceone-step real-time quantitative RT-PCR SARSPol1 system targeting polymerase (pol) gene SARS1-F forward primer
gagtgtgcgcaagtattaagtga

SEQ ID NO: 2 - DNA, 22 bp, Artificial Sequence
  Description of Artificial Sequenceone-step real-time quantitative RT-PCR SARSPol1 system targeting polymerase (pol) gene SARS1-R reverse primer
tgatgttccacctggtttaaca

SEQ ID NO: 3 - DNA, 24 bp, Artificial Sequence
  Description of Artificial SequenceSARSPol1 system targeting polymerase (pol) gene SARS1-P dual-labeled fluorescent probe
ntggtcatgtgtggcggctcactn

SEQ ID NO: 4 - DNA, 19 bp, Artificial Sequence
  Description of Artificial Sequenceone-step real-time quantitative RT-PCR SARS2-F forward primer
ccgcgaagaagctattcgt

SEQ ID NO: 5 - DNA, 20 bp, Artificial Sequence
  Description of Artificial Sequenceone-step real-time quantitative RT-PCR SARS2-R reverse primer
tgcatgacagccctctacat

SEQ ID NO: 6 - DNA, 21 bp, Artificial Sequence
  Description of Artificial SequenceSARS2-P dual-labeled fluorescent probe
ngttcgtgcgtggattggctn

SEQ ID NO: 7 - DNA, 16 bp, Artificial Sequence
  Description of Artificial Sequenceone-step real-time quantitative RT-PCR SARN system targeting nucleocapsid (nuc) gene SARS3-F forward primer
tgccctcgcgctattg

SEQ ID NO: 8 - DNA, 21 bp, Artificial Sequence
  Description of Artificial Sequenceone-step real-time quantitative RT-PCR SARN system targeting nucleocapsid (nuc) gene SARS3-R reverse primer
ggcctttaccagaaactttgc

SEQ ID NO: 9 - DNA, 24 bp, Artificial Sequence
  Description of Artificial SequenceSARSN system targeting nucleocapsid (nuc) gene SARS3-P dual-labeled fluorescent probe
ngctagacagattgaaccagcttn

SEQ ID NO: 10 - DNA, 20 bp, Artificial Sequence
  Description of Artificial Sequenceone-step real-time quantitative RT-PCR SARS4-F forward primer
ccgctcatggaaagtgaact

SEQ ID NO: 11 - DNA, 16 bp, Artificial Sequence
  Description of Artificial Sequenceone-step real-time quantitative RT-PCR SARS4-R-reverse primer
cggccattcgcaagtg

SEQ ID NO: 12 - DNA, 25 bp, Artificial Sequence
  Description of Artificial SequenceSARS4-P dual-labeled fluorescent probe
ncattggtgctgtgatcattcgtgn

SEQ ID NO: 13 - DNA, 80 bp, Artificial Sequence
  Description of Artificial Sequencesingle stranded synthetic calibrator DNA oligonucleotide SARS1-std for SARSPol1
aacgagtgtgcgcaagtattaagtgagatggtcatgtgtggcggctcactatatgttaaaccaggtggaacatcatccgg

SEQ ID NO: 14 - DNA, 72 bp, Artificial Sequence
  Description of Artificial Sequencesingle stranded synthetic calibrator DNA oligonucleotide SARS2-std
tcacccgcgaagaagctattcgtcacgttcgtgcgtggattggctttgatgtagagggctgtcatgcaacta

SEQ ID NO: 15 - DNA, 75 bp, Artificial Sequence
  Description of Artificial Sequencesingle stranded synthetic calibrator DNA oligonucleotide SARS3-std for SARSN
gaaactgccctcgcgctattgctgctagacagattgaaccagcttgagagcaaagtttctggtaaaggccaacaa

SEQ ID NO: 16 - DNA, 76 bp, Artificial Sequence
  Description of Artificial Sequencesingle stranded synthetic calibrator DNA oligonucleotide SARS4-std
accagaccgctcatggaaagtgaacttgtcattggtgctgtgatcattcgtggtcacttgcgaatggccggacact